(19) United States
(12) Patent Application Publication
(10) Pub. No.: US 2024/0262928 A1
(43) Pub. Date: Aug. 8, 2024

(54) Composition and Method

(71) Applicant: HaemaLogiX Pty Ltd, Eveleigh (AU)

(72) Inventors: Rosanne Dunn, Eveleigh (AU); Arunakumari Alahari, Eveleigh (AU)

(73) Assignee: HaemaLogiX Pty Ltd, Eveleigh (AU)

(21) Appl. No.: 17/907,471

(22) PCT Filed: Mar. 26, 2021
(86) PCT No.: PCT/IB2021/000171

§ 371 Date: Sep. 27, 2022

Related U.S. Application Data
(60) Provisional application No. 63/200,752, filed on Mar. 25, 2021.

(30) Foreign Application Priority Data
Mar. 27, 2020 (AU) 2020900948

(51) Int. Cl. C07K 16/30; C07K 1/22

(52) U.S. Cl. CPC C07K 16/3061; C07K 1/22

(57) ABSTRACT

The present disclosure relates to a method of purifying a binding protein from undesirable components. Such binding proteins may be useful for treating a disorder such as cancer.

### FIELD OF THE INVENTION

The present disclosure relates to a method of purifying a binding protein from undesirable components. Such binding proteins may be useful for treating a disorder such as cancer.

### BACKGROUND OF THE INVENTION

The economics of large-scale protein purification are important, particularly for therapeutic binding proteins, as these molecules make up a large percentage of the therapeutic biologics on the market. In addition to their therapeutic value, binding proteins such as monoclonal antibodies, for example, are also important tools in the diagnostic field. The production of binding proteins for biopharmaceutical applications typically involves the use of cell cultures that are known to produce undesirable components. While substantive progress has been made in relation to purifying binding proteins, particularly in relation to affinity chromatography, such methods may not be particularly suitable for purifying desired monomers of binding proteins. Accordingly, improved methods of purifying binding proteins are required.

### SUMMARY OF THE INVENTION

The production of binding proteins using recombinant DNA technology can often lead to the accumulation of undesirable components in the cell culture fluid. These undesirable components include high molecular weight aggregates of the binding protein and complexes of the binding protein with free light chain not associated with heavy chain. This problem may be particularly pronounced when Chinese Hamster Ovary (CHO) cells are used to produce binding proteins of interest as CHO cells naturally secrete free light chain not associated with heavy chain. The present inventors have surprisingly identified a method of purifying binding protein from free light chain not associated with heavy chain by incorporating a basic wash step into the protein purification process. The method allows higher yields of unbound binding protein to be recovered, thus providing higher purity binding protein compositions. Such compositions may be more potent therapeutically. Accordingly, in a first aspect, the present disclosure relates to a method of purifying a binding protein from free light chain not associated with heavy chain, the method comprising, loading a composition comprising the binding protein and free light chain not associated with heavy chain onto an equilibrated affinity chromatography column of neutral pH to bind the binding proteins in the composition to the affinity chromatography column; washing the affinity chromatography column with a basic wash buffer having a pH of at least 2.5 to 5 above neutral pH to wash free light chain not associated with heavy chain from the composition; and, eluting binding proteins bound to the affinity chromatography column with an elution buffer.

In an example, the composition is cell culture fluid obtained from CHO cells genetically modified to express the binding protein or a composition derived therefrom. In another example, the present disclosure encompasses a method for purifying a binding protein that binds free light chain not associated with heavy chain from a Chinese Hamster Ovary (CHO) cell culture comprising the binding protein, the method comprising:
binding the binding protein from the CHO cell culture to a Protein A resin of neutral pH;
washing the Protein A resin with a basic wash buffer having a pH of at least 2.5 to 5 above neutral pH to wash free light chain not associated with heavy chain from the composition; and
eluting binding protein bound to the Protein A resin with an elution buffer.

In an example, the binding protein comprises an antibody. In another example, the binding protein is an antibody. In another example, the antibody is an anti-kappa myeloma antigen (KMA) antibody. In another example, the antibody preferentially binds KMA over free light chain not associated with heavy chain.

In an example, the basic wash buffer has a pH of 9 to 11. In another example, the basic wash buffer has a pH of 9.5 to 10.5. In another example, the basic wash buffer comprises 0.1M to 0.2M sodium carbonate. In another example, the basic wash buffer further comprises 1M sodium chloride.

In an example, washing the affinity chromatography to wash free light chain not associated with heavy chain comprises washing the affinity chromatograph column twice with a basic wash buffer. In this example, in the first wash the basic wash buffer may comprise 0.2M sodium chloride and in the second wash the basic wash buffer may comprise 0.1M sodium chloride. In an example, the basic wash buffers have the same pH.

In another example, washing the affinity chromatography column further comprises washing with an acidic wash buffer. In an example, the acidic wash buffer has a pH of 5.5 to 6.5. In an example, the acidic wash buffer comprises about 35 mM sodium phosphate.

In an example, the elution buffer is acidic. In an example, the elution buffer has a pH lower than the acidic wash buffer. In an example, the elution buffer has a pH of 2.5 to 3.5. In an example, the elution buffer comprises about 10 mM sodium phosphate.

In an example, the affinity chromatography column is a protein A chromatography column.

In an example, the method further comprises a viral inactivation step. In another example, the method further comprises a viral filtration step. In another example, the method further comprises an ultrafiltration step.

In another example, the composition comprising the binding protein and free light chain not associated with heavy chain is cell culture fluid obtained from the cell culture of Chinese Hamster Ovary (CHO) cells which express the binding protein. In an example, the cell culture fluid has been clarified.

In another example, the free light chain not associated with heavy chain is a kappa light chain. In an example, the molecular weight of the free light chain not associated with heavy chain is about 22.5-25 kD. In another example, the free light chain not associated with heavy chain is a kappa light chain dimer. In this example, the molecular weight of the free light chain not associated with heavy chain dimer is about 45-50 kD. In an example, the molecular weight of the free light chain not associated with heavy chain or a complex thereof which is purified from a composition disclosed herein is between 20 and 100 kD. In another example, the molecular weight is between 22 and 80 kD. In another example, the molecular weight is between 22 and 50 kD.

In an example, it may be desirable to further purify compositions of the disclosure by removing high molecular weight aggregates. Accordingly, in an example, methods of the disclosure may also comprise a cation exchange chromatography step. In an example, the eluted binding protein is subject to cation exchange chromatography to remove any potential high molecular weight species. In another example, the method further comprises a step of formulating the eluted binding protein into a pharmaceutical composition or diagnostic composition.

In an example, the present disclosure encompasses a pharmaceutical or diagnostic composition which comprises a binding protein purified according to the methods disclosed herein. In an example, the pharmaceutical composition comprises a binding protein which comprises a VH region set forth in SEQ ID NO:1 and a VL region set forth in SEQ ID NO:3 or binds the same epitope of kappa myeloma antigen (KMA) as an antibody comprising a VH region set forth in SEQ ID NO:1 and a VL region set forth in SEQ ID NO:3

In an example, eluted binding proteins are at least 75% unbound antibody relative to antibody bound to free light chain not associated with heavy chain as determined by SEC-HPLC and/or BioCore assay. In another example, eluted binding proteins are at least 85% unbound binding protein relative to binding protein bound to free light chain not associated with heavy chain as determined by SEC-HPLC and/or BioCore assay. In another example, eluted binding proteins are at least 90% unbound binding protein relative to binding protein bound to free light chain not associated with heavy chain as determined by SEC-HPLC and/or BioCore assay. In another example, eluted binding proteins are between 85% and 95% unbound binding protein relative to binding protein bound to free light chain not associated with heavy chain as determined by SEC-HPLC and/or BioCore assay.

In another aspect, the present disclosure encompasses a composition comprising an anti-KMA binding protein, wherein less than 20% of the binding proteins in the composition are binding proteins in complex with free light chain not associated with heavy chain. In an example, less than 15%, less than 10%, less than 6% of the binding proteins in the composition are antibodies in complex with free light chain not associated with heavy chain. In an example, the binding proteins comprise a VH region set forth in SEQ ID NO:1 and a VL region set forth in SEQ ID NO:3 or binds the same epitope of kappa myeloma antigen (KMA) as an antibody comprising a VH region set forth in SEQ ID NO:1 and a VL region set forth in SEQ ID NO:3. In an example, the binding proteins are produced by CHO cells. In an example, the binding proteins comprise an antibody. In an example, the binding proteins are antibodies.

Any example herein shall be taken to apply mutatis mutandis to any other example unless specifically stated otherwise.

The present invention is not to be limited in scope by the specific examples described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying drawings.

### KEY TO SEQUENCE LISTING

SEQ ID NO:1—amino acid sequence of KappaMab variable heavy chain (V_{H}).
SEQ ID NO:2—KappaMab epitope amino acid sequence.
SEQ ID NO:3—amino acid sequence of KappaMab variable light chain (V_{L}).
SEQ ID NO:4—amino acid sequence of KappaMab V_{H} CDR1.
SEQ ID NO:5—amino acid sequence of KappaMab V_{H} CDR2.
SEQ ID NO:6—amino acid sequence of KappaMab V_{H} CDR3.
SEQ ID NO:7—amino acid sequence of KappaMab V_{L} CDR1.
SEQ ID NO:8—amino acid sequence of KappaMab V_{L} CDR2.
SEQ ID NO:9—amino acid sequence of KappaMab V_{L} CDR3.
SEQ ID NO:10—amino acid sequence of KappaMab epitope 2 (improved binding).

### DETAILED DESCRIPTION OF THE INVENTION

### General Techniques and Selected Definitions

Unless specifically defined otherwise, all technical and scientific terms used herein shall be taken to have the same meaning as commonly understood by one of ordinary skill in the art (e.g., molecular biology, antibody manufacture, biochemistry, oncology and protein purification).

Unless otherwise indicated, the molecular and statistical techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T.A. Brown (editor), Essential Molecular Biology: A Practical Approach, Volumes 1 and 2, IRL Press (1991), D.M. Glover and B.D. Hames (editors), DNA Cloning: A Practical Approach, Volumes 1-4, IRL Press (1995 and 1996), and F.M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J.E. Coligan et al. (editors) Current Protocols in Immunology, John Wiley & Sons (including all updates until present).

The phrase “anti-KMA binding protein” is used in the context of the present disclosure to refer to a binding protein that binds or specifically binds Kappa Myeloma Antigen. Kappa Myeloma Antigen (KMA) is a membrane-bound light chain with selectivity for kappa myeloma cells (Boux, HA. et al. (1983) J Exp Med. 158:1769).

In an example, an anti-KMA binding protein is capable of binding KMA bearing cells. In another example, the anti-KMA binding protein is capable of killing KMA bearing cells. In an example, anti-KMA binding proteins encompassed by the present disclosure do not bind intact immunoglobulin. Put another way, exemplary anti-KMA binding proteins do not recognise kappa light chains that are in association with Ig heavy chain such as in intact Ig molecules.

As used herein, the term “binds” in reference to the interaction of a binding protein described herein and KMA means that the interaction is dependent upon the presence of a particular structure (e.g., an antigenic determinant or epitope) on KMA. For example, a binding protein recognizes and binds to a specific antigen structure rather than to antigens generally. For example, if a binding protein binds to epitope “A”, the presence of a molecule containing epitope “A” (or free, unlabelled “A”), in a reaction containing labelled “A” and the binding protein, will reduce the amount of labelled “A” bound to the binding protein. In an example, a KMA binding protein disclosed herein preferentially binds KMA (i.e. cell surface antigen) over free kappa light chain (e.g. serum antigen). A binding protein disclosed herein that preferentially binds KMA over free kappa light chain reacts or associates more frequently, more rapidly, with greater duration and/or with greater affinity with KMA than it does with free light chain.

As used herein, the term “specifically binds” shall be taken to mean that the binding interaction between a binding protein and KMA is dependent on detection of the KMA by the binding protein. Accordingly, the binding protein specifically binds or recognizes KMA even when present in a mixture of other molecules, cells or organisms. In one example, the binding protein reacts or associates more frequently, more rapidly, with greater duration and/or with greater affinity with KMA than it does with alternative antigens or cells. In an example, a binding protein disclosed herein that specifically binds KMA can also preferentially bind or recognize KMA over free light chain. It is also understood by reading this definition that, for example, a binding protein that specifically binds to KMA may or may not specifically bind to a second antigen. As such, “specific binding” does not necessarily require exclusive binding or non-detectable binding of another antigen. The term “specifically binds” can be used interchangeably with “selectively binds” herein. Generally, reference herein to binding means specific binding, and each term shall be understood to provide explicit support for the other term. Methods for determining specific binding will be apparent to the skilled person. For example, a binding protein of the disclosure is contacted with KMA or an alternative antigen. Binding of the binding protein to KMA or alternative antigen is then determined and a binding protein that binds as set out above to the KMA rather than the alternative antigen is considered to specifically bind to KMA. A similar method may be used to identify preferential binding. In this instance, the alternative antigen would be free light chain.

The term “immunoglobulin” will be understood to include binding proteins of the disclosure, such as anti-KMA binding proteins, which comprise an immunoglobulin domain. Exemplary immunoglobulins are antibodies. Additional proteins encompassed by the term “immunoglobulin” include domain antibodies, camelid antibodies and antibodies from cartilaginous fish (i.e., immunoglobulin new antigen receptors (IgNARs)). Generally, camelid antibodies and IgNARs comprise a V_{H}, however lack a V_{L} and are often referred to as heavy chain immunoglobulins. Other “immunoglobulins” include T cell receptors.

The term “binding protein” is used in the context of the present disclosure to refer to human or humanised immunoglobulin molecules immunologically reactive with a particular antigen and includes both polyclonal and monoclonal antibodies. The term “binding protein” also includes antigen binding forms of antibodies, including fragments with antigen-binding capability (e.g., Fab′, F(ab′)₂, Fab, Fv and rIgG as discussed in Pierce Catalogue and Handbook, 1994-1995 (Pierce Chemical Co., Rockford, Ill.); Kuby, J., Immunology, 3^{rd} Ed., W.H. Freeman & Co., New York (1998). The term is also used to refer to recombinant single chain Fv fragments (scFv) as well as divalent (di-scFv) and trivalent (tri-scFV) forms thereof. The term antibody also includes diabodies, triabodies, and tetrabodies. In an example, binding proteins of the disclosure bind free light chain not associated with heavy chain. In another example, binding proteins bind free kappa light chain not associated with heavy chain.

The term binding protein as used herein encompasses binding proteins which comprise an antibody such as a bi-specific molecule. For example, a binding protein may comprise an above referenced immunoglobulin such as an antibody and an above referenced fragment such as an Fv.

An “antigen binding fragment” of an antibody comprises one or more variable regions of an intact antibody. Examples of antibody fragments include Fab, Fab′, F(ab′)2 and Fv fragments; diabodies; linear antibodies and single-chain antibody molecules formed from antibody fragments. For example, the term antigen binding fragment may be used to refer to recombinant single chain Fv fragments (scFv) as well as divalent (di-scFv) and trivalent (tri-scFV) forms thereof. In an example, the binding protein is an antigen binding fragment. Such fragments can be produced via various methods known in the art.

The term “complementarity determining region” or “CDR” is used in the context of the present disclosure to refer to the part of the two variable chains of antibodies (heavy and light chains) that recognize and bind to the particular antigen. The CDRs are the most variable portion of the variable chains and provide binding proteins with their specificity. There are generally three CDRs on each of the variable heavy (V_{H}) and variable light (V_{L}) chains.

As used herein, “variable region” refers to the portions of the light and/or heavy chains of an antibody as defined herein that specifically binds to an antigen and, for example, includes amino acid sequences of CDRs; i.e., CDR1, CDR2, and CDR3, and framework regions (FRs). For example, the variable region comprises three or four FRs (e.g., FR1, FR2, FR3 and optionally FR4) together with three CDRs. V_{H} refers to the variable region of the heavy chain. V_{L} refers to the variable region of the light chain.

In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system” or “Kabat”.

Other conventions that include corrections or alternate numbering systems for variable domains include IMGT (Lefranc, et al. (2003), Dev Comp Immunol 27: 55-77), Chothia (Chothia C, Lesk AM (1987), J Mal Biol 196: 901-917; Chothia, et al. (1989), Nature 342: 877-883) and AHo (Honegger A, Plückthun A (2001) J Mol Biol 309: 657-670). For convenience, examples of binding proteins of the present disclosure may also be labelled according to IMGT.

The term “antibody heavy chain” is used herein to refer to the larger of the two types of polypeptide chains present in all antibody molecules in their naturally occurring conformations. An “antibody light chain,” as used herein, refers to the smaller of the two types of polypeptide chains present in all antibody molecules in their naturally occurring conformations. κ and λ light chains refer to the two major antibody light chain isotypes.

Terms such as “host cell,” “host cell line,” and “host cell culture” are used interchangeably in the context of the present disclosure to refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. In an example, the host cell is a Chinese Hamster Ovary (CHO) cell.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill of those practicing in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

A “buffer” refers to a substance which, by its presence in solution, increases the amount of acid or alkali that must be added to cause unit change in pH. A buffered solution resists changes in pH by the action of its acid-base conjugate components. Buffered solutions for use with biological reagents are generally capable of maintaining a constant concentration of hydrogen ions such that the pH of the solution is within a physiological range. Traditional buffer components include, but are not limited to, organic and inorganic salts, acids and bases. Exemplary buffers for use in purification of biological molecules (e.g., antibodies) include the zwitteronic or “Good” Buffers, see e.g., Good et al. (1966) Biochemistry 5:467 and Good and Izawa (1972) Methods Enzymol. 24:62. Exemplary buffers include but are not limited to TES, MES, PIPES, HEPES, MOPS, MOPSO, TRICINE and BICINE.

The term “wash buffer” is used herein to refer to a solution used to carry away impurities such as free light chain not associated with heavy chain from a given material, e.g., composition or column or resin, to which a binding protein disclosed herein is bound.

The term “basic” is used in the context of the present disclosure to refer to buffers having a basic pH. For example, the term may be used in relation to wash buffers to refer to wash buffers having a basic pH. In an example, the term basic is used to refer to wash buffers having a pH at least 2.5 above neutral. In another example, the term basic is used to refer to wash buffers having a pH at least 3 above neutral. In another example, the term basic is used to refer to wash buffers having a pH at least 3.5 above neutral. In another example, the term basic is used to refer to wash buffers having a pH at least 4 above neutral. In another example, the term basic is used to refer to wash buffers having a pH at least 4.5 above neutral. In another example, the term basic is used to refer to wash buffers having a pH at least 4.5 above neutral. In another example, the term basic is used to refer to wash buffers having a pH at least 5 above neutral. In another example, the term basic is used to refer to wash buffers having a pH at least 5.5 above neutral. In another example, the term basic is used to refer to wash buffers having a pH between 2.5 and 5.5 above neutral. In another example, the term basic is used to refer to wash buffers having a pH between 3 and 5 above neutral. In an example, a basic wash buffer has a pH between 9 and 11. In an example, a basic wash buffer has a pH between 9.5 and 10.5. In an example, a basic wash buffer has a pH of at least 9. In an example, a basic wash buffer has a pH of at least 9.5.

In contrast, the term “acidic” is used in the context of the present disclosure to refer to buffers having an acidic pH. For example, the term may be used in relation to wash buffers to refer to wash buffers having an acidic pH. Acidic buffers disclosed herein have a pH less than 7. In an example, an acidic wash buffer has a pH between 5 and 6.5. In an example, an acidic wash buffer has a pH between 5.5 and 6.5. In an example, an acidic wash buffer has a pH less than or equal to 6.5. Other buffers of the disclosure such as elution buffers may be more acidic than wash buffers. For example, an elution buffer disclosed herein may have a pH less than 4. In another example, an elution buffer can have a pH less than or equal to 3.5. In another example, an elution buffer can have a pH between 2.5 and 3.5.

As used herein, the term “neutral pH” refers to pH of 7.

As used herein, the term “affinity chromatography column” refers to a column comprising the resin by which affinity chromatography is performed. In one example, the affinity chromatography comprises subjecting a composition disclosed herein to a column comprising a suitable affinity chromatographic support. Non-limiting examples of such chromatographic supports include, but are not limited to Protein A resin, Protein G resin, affinity supports comprising the antigen against which the binding protein of interest was raised, and affinity supports comprising an Fc binding protein. For example, the affinity chromatography column may comprise a protein A chromatography resin, protein L chromatography resin or protein G chromatography resin. In an example, the affinity chromatography column comprises a protein A chromatography resin. Commercially available examples of protein A chromatography resins include MabSelect Xtra and MabSelect SuRe.

As used herein, the term “viral inactivation step” refers to a process by which viruses remain in a composition, but have been rendered permanently non-viable. For example, viral inactivation may be effected by adjusting the solution to low pH.

As used herein, the term “low pH” shall be understood to mean a pH between 2 and 4, or a pH between 3.4 and 3.6, or a pH of 3.5.

As used herein, the term “viral filtration step” refers to a process by which viruses are removed from a composition. For example, viral filtration may be effected by passing the composition through a filter such as a nanofilter (e.g. Planova 20N).

As used herein, the term “high molecular weight form” or “high molecular weight forms” refers to binding proteins in a form of two or more binding protein monomers. For example, high molecular weight forms of binding protein (e.g. anti-KMA binding protein) is a dimer, or a trimer, or tetramer.

As used herein, the term “host cell” refers to any cell that is capable of expressing recombinant binding protein disclosed herein, including bacteria, insect and mammalian cells. For example, mammalian cells may be a HEK293 cells or Chinese hamster ovary cells (CHO cells). In an example, the host cell is a CHO cell. For example, the host cell can be a genetically modified host cell that expresses a binding protein disclosed herein. Accordingly, in an example, the methods of the present disclosure may be used to purify binding proteins disclosed herein from culture fluid of CHO cell culture or a composition derived therefrom.

As used herein, the term “fermentation” refers to a process where a host cell containing a polynucleotide sequence encoding a binding protein is propagated in cell culture medium to express the binding protein. Optimal fermentation conditions are dependent on a number of parameters which include, but not limited to, temperature range, aeration level, feed rate and media composition. Fermentation may be performed under aerobic, anaerobic or microaerobic conditions. Fermentation may also be performed in large scale batch culture using for example one or bioreactors.

As used herein, the term “clarified” or “clarification” refers to one or more steps involving removal of whole cells and/or cellular debris using one or more steps including any of the following alone or in combination: centrifugation, depth filtration, precipitation, flocculation and/or settling. Clarification generally involves the removal of one or more impurities and performed prior to a purification step involving capture of binding protein. For example, clarification may involve depth filtration and centrifugation. In an example, compositions of the disclosure are clarified before being purified according to the methods disclosed herein.

As used herein, the term “cell culture fluid” refers to the cell culture medium comprising binding protein during or following fermentation but before a purification step involving the capture of the binding protein. In an example, the cell culture fluid has been purified or partially purified to provide a preparation comprising binding protein and free light chain not associated with heavy chain.

The term “purify” or “purifying” or “purification” refers to the removal, whether completely or partially, of at least one impurity from a solution containing binding protein and one or more impurities, which thereby improves the level of purity of the binding protein in the solution. Impurities include DNA, RNA, host cell protein (HCP), endotoxins, lipids, and one or more additives which may be present with the binding protein as a result of methods of the present disclosure e.g. produced by a step performed before or during the purification process. The binding protein which is purified is preferably essentially pure and desirably essentially homogeneous (i.e. free from contaminating proteins etc.). In an example, the methods of the present disclosure purify or partially purify free light chain not associated with heavy chain from compositions disclosed herein. In another example, the methods of the present disclosure purify or partially purify free light chain not associated with heavy chain and high molecular weight aggregates from compositions disclosed herein. In an example, the high molecular weight aggregates are binding protein complexes such as dimers. In an example, a purified binding protein is at least 60% free, more preferably at least 75% free, and more preferably at least 90% free from free light chain not associated with heavy chain. In another example, a purified binding protein is at least 60% free, more preferably at least 75% free, and more preferably at least 90% free from free light chain not associated with heavy chain and high molecular weight aggregates of the binding protein. In these examples, the free light chain not associated with heavy chain can be kappa light chain. In an example, the free light chain not associated with heavy chain has a molecular weight between 22 and 25 kD. In an example, the free light chain not associated with heavy chain is a kappa light chain dimer. In an example, the free light chain not associated with heavy chain has a molecular weight between 45 and 50 kD.

As used herein, the term “pharmaceutical composition” refers to a formulation of binding protein with compounds generally accepted in the art for the delivery of therapeutic proteins to humans. Exemplary compounds include all pharmaceutically acceptable carriers, diluents or excipients thereof.

As used herein, the term “diagnostic composition” refers to a formulation of binding protein with compounds generally accepted in the art for provision of binding proteins disclosed herein in a diagnostic form. Such formulations may be used in vitro or in vivo and therefore, depending on the application, can be formulated accordingly with the appropriate carriers, diluents and/or excipients.

As used in this specification and the appended claims, terms in the singular and the singular forms “a,” “an” and “the,” for example, optionally include plural referents unless the content clearly dictates otherwise.

As used herein, the term “about”, unless stated to the contrary, refers to +/−10%, more preferably +/−5%, more preferably +/−1%, of the designated value.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Binding Proteins

Binding proteins for production, purification, formulation or use in the present disclosure include, but are not limited to the following disclosures. In an example, the binding protein is a recombinant binding protein. In an example, the binding proteins are produced by CHO cells.

In an example, the binding protein can comprise an antibody. For example, the binding protein can be an antibody. For example, the binding protein can be a monoclonal antibody. In an example, the binding protein is a human antibody. In an example, the antibody is humanized. In an example, the antibody is a chimeric antibody.

In an example, the binding protein is an anti-KMA binding protein. For example, the binding protein can be an anti-KMA antibody. In an example, the anti-KMA binding protein preferentially binds KMA over free light chain not associated with heavy chain.

In an example, the binding protein comprises a VH region and a VL region, wherein the VH region comprises a CDR1 which comprises the amino acid sequence set forth in SEQ ID NO:4, a CDR2 which comprises the amino acid sequence set forth in SEQ ID NO:5 and a CDR3 which comprises the amino acid sequence set forth in SEQ ID NO:6 and, wherein the VL region comprises a CDR1 which comprises the amino acid sequence set forth in SEQ ID NO:7, a CDR2 which comprises the amino acid sequence set forth in SEQ ID NO:8 and a CDR3 which comprises the amino acid sequence set forth in SEQ ID NO:9. In this example, the binding protein can comprise an antibody. For example, the binding protein may be a bi-specific molecule which comprises an antibody. In this example, the antibody can comprise the above referenced CDRs. In another example, the binding protein is an antibody.

Accordingly, in an example, the binding protein is an antibody comprising a VH region and a VL region, wherein the VH region comprises a CDR1 which comprises the amino acid sequence set forth in SEQ ID NO:4, a CDR2 which comprises the amino acid sequence set forth in SEQ ID NO:5 and a CDR3 which comprises the amino acid sequence set forth in SEQ ID NO:6 and, wherein the VL region comprises a CDR1 which comprises the amino acid sequence set forth in SEQ ID NO:7, a CDR2 which comprises the amino acid sequence set forth in SEQ ID NO:8 and a CDR3 which comprises the amino acid sequence set forth in SEQ ID NO:9.

In another example, the binding protein is an antibody comprising a VH region which comprises an amino acid sequence at least 80% identical to the amino acid sequence set forth in SEQ ID NO:1 and a VL region which comprises an amino acid sequence at least 80% identical to the amino acid sequence set forth in SEQ ID NO:3 or binds the same epitope of kappa myeloma antigen (KMA) as an antibody comprising a VH region set forth in SEQ ID NO:1 and a VL region set forth in SEQ ID NO:3.

In another example, the binding protein is an antibody comprising a VH region which comprises an amino acid sequence at least 90% identical to the amino acid sequence set forth in SEQ ID NO: 1 and a VL region which comprises an amino acid sequence at least 90% identical to the amino acid sequence set forth in SEQ ID NO:3 or binds the same epitope of kappa myeloma antigen (KMA) as an antibody comprising a VH region set forth in SEQ ID NO:1 and a VL region set forth in SEQ ID NO:3.

In another example, the binding protein is an antibody comprising a VH region which comprises an amino acid sequence at least 95% identical to the amino acid sequence set forth in SEQ ID NO:1 and a VL region which comprises an amino acid sequence at least 95% identical to the amino acid sequence set forth in SEQ ID NO:3 or binds the same epitope of kappa myeloma antigen (KMA) as an antibody comprising a VH region set forth in SEQ ID NO:1 and a VL region set forth in SEQ ID NO:3.

In another example, the binding protein is an antibody comprising a VH region which comprises an amino acid sequence at least 99% identical to the amino acid sequence set forth in SEQ ID NO:1 and a VL region which comprises an amino acid sequence at least 99% identical to the amino acid sequence set forth in SEQ ID NO:3 or binds the same epitope of kappa myeloma antigen (KMA) as an antibody comprising a VH region set forth in SEQ ID NO:1 and a VL region set forth in SEQ ID NO:3.

In another example, the binding protein is an antibody comprising a VH region set forth in SEQ ID NO:1 and a VL region set forth in SEQ ID NO:3 or binds the same epitope of kappa myeloma antigen (KMA) as an antibody comprising a VH region set forth in SEQ ID NO:1 and a VL region set forth in SEQ ID NO:3.

In another example, the binding protein is an antibody comprising a VH region set forth in SEQ ID NO:1 and a VL region set forth in SEQ ID NO:3.

In various examples, above referenced sequence variants having recited % identity with the recited SEQ IDs can also have a VH region and a VL region, wherein the VH region comprises a CDR1 which comprises the amino acid sequence set forth in SEQ ID NO:4, a CDR2 which comprises the amino acid sequence set forth in SEQ ID NO:5 and a CDR3 which comprises the amino acid sequence set forth in SEQ ID NO:6 and, wherein the VL region comprises a CDR1 which comprises the amino acid sequence set forth in SEQ ID NO:7, a CDR2 which comprises the amino acid sequence set forth in SEQ ID NO:8 and a CDR3 which comprises the amino acid sequence set forth in SEQ ID NO:9. For example, the anti-KMA binding protein has a VH comprising CDRs as shown in SEQ ID NO: 4, SEQ ID NO: 5, SEQ ID NO: 6, an amino acid sequence at least 90%, at least 95%, at least 98%, at least 99% identical to SEQ ID NO: 1 and a VL comprising CDRs as shown in SEQ ID NO: 7, SEQ ID NO: 8, SEQ ID NO: 9 and an amino acid sequence at least 90%, at least 95%, at least 98%, at least 99% identical to SEQ ID NO: 3.

In another example, the anti-KMA binding protein has the CDRs shown in SEQ ID NO: 1 and SEQ ID NO: 3, wherein the CDRs are assigned using the Kabat numbering system. In another example, the anti-KMA binding protein has the CDRs shown in SEQ ID NO: 1 and SEQ ID NO: 3, wherein the CDRs are assigned using the IMGT numbering system. In another example, the anti-KMA binding protein has the CDRs shown in SEQ ID NO: 1 and SEQ ID NO: 3, wherein the CDRs are assigned using EU numbering system of Kabat.

In an example, the anti-KMA binding protein is a naked antibody. In other examples, the anti-KMA binding protein is a full-length antibody, intact antibody or whole antibody. In an example, the anti-KMA binding protein is monospecific. In an example, the anti-KMA binding protein is bi-specific.

Furthermore, in the above examples, the binding protein can bind a KMA epitope which comprises the amino acid sequence set forth in SEQ ID NO:2 or SEQ ID NO:10.

In another example, an anti-KMA binding protein according to the present disclosure competes with an antibody that binds or specifically binds an epitope comprising an amino acid sequence as shown in SEQ ID NO: 2. In another example, an anti-KMA binding protein according to the present disclosure competes with an antibody that binds or specifically binds an epitope consisting of the amino acid sequence as shown in SEQ ID NO: 10.

Binding proteins may be identified by their ability to compete for binding to KMA or a region or epitope thereof using various methods known in the art. For example, binding to KMA on kappa human myeloma cell lines (κHMCL) such as KMS-11, KMS-26 and JJN3 can be assessed (Asvadi et al. (2015) British Journal of Haematology, 169, 333-343). In this procedure, an anti-KMA binding is conjugated with biotin using established procedures (Hofmann K, et al. (1982) Biochemistry 21: 978-84). Binding proteins are then evaluated by their capacity to compete with the binding of the biotinylated antibody to KMA on κHMCL cells. The binding of biotinylated antibody to κHMCL cells may be assessed by the addition of fluorescein-labelled streptavidin which will bind to biotin on the labelled antibody. Fluorescence staining of cells is then quantified by flow cytometry, and the competitive effect of antibodies expressed as a percentage of the fluorescence levels obtained in the absence of the competitor.

In one example, the binding protein comprises an immune cell engager. For example, the binding protein can be an immune cell engaging bi-specific binding protein. For example, the immune cell engager can engage a binding protein disclosed herein to T-cells or Natural Killer (NK) cells. Examples of immune cell engagers include anti-CD3 binding domains, anti-CD19 binding domains and anti-CD16 binding domains. In an example, the immune cell engager can engage T-cells via an anti-CD3 binding domain. In other examples, the immune cell engager can engage T-cells via an anti-CD4 or anti-CD8 binding domain. Various other examples of immune cell engagers are disclosed in Suurs et al., (2019) Pharmacology and Therapeutics., 201:103-119.

In the above referenced examples, the affinity of a binding protein disclosed herein for KMA can be measured using various methods. In an example, the dissociation constant (K_{D}) or association constant (K_{A}) or equilibrium constant (K_{D}) of a binding protein for KMA is determined. These constants for a binding protein are, in one example, measured by a radiolabelled or fluorescently-labelled KMA-binding assay. This assay equilibrates the binding protein with a minimal concentration of labelled KMA in the presence of a titration series of unlabelled KMA. Following washing to remove unbound KMA, the amount of label is determined. Similar assays may be performed using an amino acid sequence which comprises SEQ ID NO:2.

Affinity measurements can be determined by standard methodology for antibody reactions, for example, immunoassays, surface plasmon resonance (SPR) (Rich and Myszka Curr. Opin. Biotechnol 11:54, 2000; Englebienne Analyst. 123: 1599, 1998), isothermal titration calorimetry (ITC) or other kinetic interaction assays known in the art. In one example, the constants are measured by using surface plasmon resonance assays, e.g., using BIAcore surface plasmon resonance (BIAcore, Inc., Piscataway, NJ) with immobilized LMA. Exemplary SPR methods are described in U.S. Pat. No. 7,229,619.

### Binding Protein Compositions

The present inventors have surprisingly identified useful binding protein compositions which comprise low levels of binding protein bound to free light chain not associated with heavy chain. Such compositions may be particularly advantageous because of increased therapeutic potency resulting in more effective treatment or, potentially, lower dosing and therefore increased safety and/or more cost effective manufacture. In an example, the present disclosure relates to a composition which comprises an above referenced binding protein wherein less than 20% of the binding proteins in the composition are binding proteins in complex with free light chain not associated with heavy chain. In another example, the composition comprises an above referenced binding protein wherein less than 15% of the binding proteins in the composition are binding proteins in complex with free light chain not associated with heavy chain. In another example, the composition comprises an above referenced binding protein wherein less than 10% of the binding proteins in the composition are binding proteins in complex with free light chain not associated with heavy chain. In another example, the composition comprises an above referenced binding protein wherein less than 6% of the binding proteins in the composition are binding proteins in complex with free light chain not associated with heavy chain. In an example, the binding protein is an anti-KMA binding protein. In an example, the anti-KMA binding protein comprises a VH and a VL, wherein the VH comprises CDRs as set forth in SEQ ID Nos: 4, 5 and 6 and the VL comprises CDRs as set forth in SEQ ID Nos: 7, 8 and 9. In an example, the anti-KMA binding protein comprises a VH comprising the amino acid sequence set forth in SEQ ID NO:1 and a VL comprising the amino acid sequence set forth in SEQ ID NO:3. In an example, the binding proteins in the composition are produced by CHO cells. In an example, the binding proteins in the composition comprise an antibody. In an example, the binding proteins in the composition are antibodies.

### Production of Binding Proteins

In one example, a binding protein as described herein is a peptide or polypeptide (e.g., is an antibody or antigen binding fragment thereof). In one example, the binding protein is recombinant.

In the case of a recombinant peptide or polypeptide, nucleic acid encoding same can be cloned into expression vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce immunoglobulin or antibody protein.

Suitable molecular cloning techniques are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art. See U.S. Pat. Nos. 4,816,567 or 5,530,101.

Following isolation, the nucleic acid is inserted operably linked to a promoter in an expression construct or expression vector for further cloning (amplification of the DNA) or for expression in a cell-free system or in cells. Thus, another example of the disclosure provides an expression construct that comprises an isolated nucleic acid of the disclosure and one or more additional nucleotide sequences. Suitably, the expression construct is in the form of, or comprises genetic components of, a plasmid, bacteriophage, a cosmid, a yeast or bacterial artificial chromosome as are understood in the art. Expression constructs may be suitable for maintenance and propagation of the isolated nucleic acid in bacteria or other host cells, for manipulation by recombinant DNA technology and/or for expression of the nucleic acid or a binding protein of the disclosure.

Many vectors for expression in cells are available. The vector components generally include, but are not limited to, one or more of the following: a signal sequence, a sequence encoding the binding protein (e.g., derived from the information provided herein), an enhancer element, a promoter, and a transcription termination sequence. Exemplary signal sequences include prokaryotic secretion signals (e.g., pe1B, alkaline phosphatase, penicillinase, Ipp, or heat-stable enterotoxin II), yeast secretion signals (e.g., invertase leader, α factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin or antibody promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the binding protein (e.g., antibody or antigen binding fragment) may be cultured in a variety of media, depending on the cell type used. Commercially available media such as Ham's F10 (Sigma), Minimal Essential Medium ((MEM), (Sigma), RPM1-1640 (Sigma), and Dulbecco's Modified Eagle's Medium ((DMEM), Sigma) are suitable for culturing mammalian cells. Media for culturing other cell types discussed herein are known in the art.

An exemplary protocol for the production of binding proteins may include the following steps. Mammalian host cells capable of expressing recombinant binding protein may be cultured in a stirred tank bioreactor system and a fed-batch culture. In an example fed-batch culture, the mammalian host cells and culture medium are supplied to a culturing vessel initially and additional culture nutrients are fed, continuously or in discrete increments, to the culture during culturing, with or without periodic removal of cell and/or binding protein from the culturing vessel before termination of fermentation.

In the growth phase, mammalian host cells are grown under conditions and for a period of time that is optimised for growth. Culture conditions, such as temperature, pH, dissolved oxygen (dO₂), and nutrient supplementation are generally tailored according to host cell and will be apparent to the ordinary skilled person. Generally, the pH is adjusted to a level between about 6.5 and 7.5. A suitable temperature range for culturing mammalian cells such as CHO cells is between about 30° C. to 38° C., and a suitable dO₂ is between 5-90% of air saturation. The cell culture environment during the production phase of the fermentation is typically controlled to ensure quality and consistency in production of binding protein between batches. Typically, binding proteins produced by CHO cells are secreted into the cell culture media. Following fermentation, the cell culture medium comprising binding proteins may be clarified.

### Free Light Chain-binding Protein Complexes

The present inventors experimentally identified that during production of binding proteins, free light chain not associated with heavy chain can bind and form complexes with binding proteins. Presence of such complexes in therapeutic formulations are particularly undesirable as they may affect the potency of the formulations.

In one example, the present disclosure relates to a method of purifying a binding protein from free light chain not associated with heavy chain comprising loading a composition comprising the binding protein and free light chain not associated with heavy chain onto an equilibrated affinity column of neutral pH to bind the binding proteins in the composition to the affinity chromatography column, washing the affinity chromatography column with a basic wash buffer having a pH of at least 2.5 to 5 above neutral pH to wash free light chain not associated with heavy chain from the composition; and eluting the binding protein bound to the affinity column with an elution buffer.

In an example, “equilibrating”, “washing” or “eluting” comprises at least 1 column volume (CV) of a respective buffer being passed through the chromatography column. For example, a “wash” step may comprise at least 1 CV of wash buffer being passed through the chromatography column. In another example, at least 1 to 25 CV of wash buffer is passed through the chromatography column in a wash step. In another example, at least 3 to 20 CV of wash buffer is passed through the chromatography column in a wash step. In another example, at least 5 to 15 CV of wash buffer is passed through the chromatography column in a wash step. In an example, an “elution” or “eluting” step may comprise at least 1 CV of elution buffer being passed through the chromatography column. In another example, at least 1 to 25 CV of elution buffer is passed through the chromatography column in an elution step. In another example, at least 3 to 20 CV of elution buffer is passed through the chromatography column in an elution step. In another example, at least 5 to 15 CV of elution buffer is passed through the chromatography column in an elution step. Determining the number of CVs required in each step is considered well within the purview of those of skill in the art.

In an example, the affinity chromatography column is equilibrated with 1 X phosphate buffered saline (PBS). For example, the affinity chromatography column can be equilibrated with at least 10 CVs of 1 X PBS.

As used herein, the term “wash buffer” refers to a buffer formulated to displace free light chain not associated with heavy chain from the solid phase of the chromatography column. In an example, the method comprises washing with a basic wash buffer having a pH of at least 2.5 to 5 above neutral pH. In an example, the basic was buffer has a pH of 9.5 to 10.5. In another example, the basic wash buffer comprises sodium carbonate. Exemplary concentrations of sodium carbonate in the wash buffer range from 0.1M to 0.2M. In another example, the basic wash buffer also comprises sodium chloride. In an example, the basic wash buffer comprises 1M sodium chloride.

In an example, washing the affinity chromatography column comprises two washes with a basic wash buffer. In other examples, the affinity chromatography column can be washed, three, four or five times with a basic wash buffer before binding proteins bound to the column are eluted. In an example, the basic wash buffers have the same pH. For example, the wash buffers can have a pH of 10.2. In an example, the column is washed with a basic wash buffer which comprises 0.2M sodium carbonate before being washed with another basic wash buffer which comprises 0.1M sodium carbonate. In this example about 5-20 CV of each wash buffer can be passed through the chromatography column. In an example, at least 10 CVs are passed through the chromatography column in the first wash and at least 5 CVs are passed through the chromatography column in the second wash.

In an example, methods of the present disclosure comprise washing the affinity chromatography column with an acidic wash buffer. In an example, the methods of the present disclosure comprise washing the affinity chromatography column with an acidic wash buffer after the column has been washed with a basic wash buffer. In an example, the acidic wash buffer has a pH of 5.5 to 6.5. In an example, the acidic wash buffer comprises sodium phosphate. For example, the acidic wash buffer can comprise 20-50 mM sodium phosphate. In an example, the acidic wash buffer can comprise about 35 mM sodium phosphate.

In an example, binding protein bound to the affinity chromatography column can be eluted by washing the affinity chromatography column with an elution buffer. Accordingly, as used herein, the term “elution buffer” refers to a buffer formulated to remove binding protein bound to the chromatography column. The elution buffer acts to dissociate the binding protein. Typical elution substances are well known in the art and may have higher concentrations of salts, free affinity ligands or analogues, or other substances that promote dissociation of the binding protein from the given material. The conductivity and/or pH of the elution buffer is/are such that the binding protein is eluted from the column. In an example, the elution buffer is acidic. In an example, the elution buffer is more acidic than a wash buffer used in a method disclosed herein. In an example, the elution buffer has a pH of 2-4. In an example, the elution buffer has a pH of 2.5 to 3.5. In an example, the elution buffer has a pH of 3. In an example, the elution buffer comprises sodium phosphate. For example, the elution buffer can comprise between 5 and 15 mM sodium phosphate. In an example, the elution buffer comprises 10 mM sodium phosphate.

In an example, binding protein eluted from the affinity chromatography column is 75% or 85%, or 90%, or 95%, or 99% unbound binding protein relative to binding protein bound to free light chain not associated with heavy chain as determined by size exclusion chromatography HPLC (SEC-HPLC) and/or BioCore Assay.

For example, binding protein eluted from the affinity chromatography column is at least 75% unbound binding protein relative to binding protein bound to free light chain not associated with heavy chain as determined by SEC-HPLC and/or BioCore Assay.

In an example, binding protein eluted from the affinity chromatography column is at least 85% unbound binding protein relative to binding protein bound to free light chain not associated with heavy chain as determined by SEC-HPLC and/or BioCore Assay.

In an example, binding protein eluted from the affinity chromatography column of at least 90% unbound binding protein relative to binding protein bound to free light chain not associated with heavy chain as determined by SEC-HPLC and/or BioCore Assay.

In an example, binding protein eluted from the affinity chromatography column of at least 95% unbound binding protein relative to binding protein bound to free light chain not associated with heavy chain as determined by SEC-HPLC and/or BioCore Assay.

In an example, binding protein eluted from the affinity chromatography column is between 85% and 95% unbound binding protein relative to binding protein bound to free light chain not associated with heavy chain as determined by SEC-HPLC and/or BioCore Assay.

In an example, binding protein eluted from the affinity chromatography column is between 90% and 95% unbound binding protein relative to binding protein bound to free light chain not associated with heavy chain as determined by SEC-HPLC and/or BioCore Assay.

In another example, the disclosure provides a method for purifying a binding protein that binds light chain not associated with heavy chain from Chinese Hamster Ovary (CHO) cell culture expressing the binding protein, the method comprising binding the binding protein from the CHO cell culture or a composition derived therefrom to an affinity chromatography resin of neutral pH; washing the resin with a basic wash buffer having a pH of at least 2.5 to 5 above neutral pH; and eluting the binding protein bound to the resin with an elution buffer. In an example, the affinity chromatography column comprises a protein A resin.

In an example, the free light chain not associated with heavy chain removed using the methods of the present disclosure is a kappa light chain. In an example, the molecular weight of the free light chain not associated with heavy chain removed using the methods of the present disclosure is between 22 and 25 kD. In another example, the free light chain not associated with heavy chain removed using the methods of the present disclosure is a kappa light chain dimer. In an example, the molecular weight of the free light chain not associated with heavy chain removed using the methods of the present disclosure is between 45 and 50 kD.

In an example, the molecular weight of the free light chain not associated with heavy chain or a complex thereof which is purified from a composition disclosed herein is between 20 and 100 kD. In another example, the molecular weight is between 22 and 80 kD. In another example, the molecular weight is between 22 and 50 kD.

In an example, the affinity chromatography step comprises subjecting the composition to a column comprising a suitable affinity chromatographic support. Non-limiting examples of such chromatographic supports include, but are not limited to Protein A resin, Protein G resin, affinity supports comprising the antigen against which the antibody of interest was raised, and affinity supports comprising an Fc binding protein. Protein A resin is useful for binding antibodies (IgG). In one example, the affinity chromatography column comprises a Protein A resin. In an example, the affinity chromatography column is a protein A chromatography column. In another example, the affinity chromatography column is a protein L chromatography column. In a further example, the affinity chromatography column is a protein G chromatography column.

The eluate can be monitored using techniques well known to those skilled in the art. For example, the absorbance at OD₂₈₀ can be followed. Eluted binding proteins can be prepared for further processing via one or more of the additional method steps discussed below if required.

### Starting Composition

In an example, the starting composition is cell culture fluid obtained following culture of cells which have been genetically modified to express a binding protein disclosed herein. For example, the starting composition can be cell culture fluid obtained following culture of CHO cells which have been genetically modified to express a binding protein disclosed herein. However, this starting composition may need to be partially purified before being subject to the methods of the present disclosure. For example, the cell culture fluid may need to be clarified to remove cell debris. Accordingly, the compositions purified according to the methods of the present disclosure are not particularly restricted so long as they are derived from cell culture fluid obtained following culture of cells which have been genetically modified to express a binding protein disclosed herein and comprise binding protein and free light chain not associated with heavy chain. In an example, the starting composition comprises unbound binding protein, binding protein bound to free light chain not associated with heavy chain and unbound free light chain not associated with heavy chain. In an example, the composition also comprises high molecular weight aggregates of the binding protein.

In an example, the starting composition is derived from cell culture fluid obtained following culture of CHO cells which have been genetically modified to express a binding protein disclosed herein such as an anti-KMA binding protein.

### Additional Method Steps

In an example, the methods of the present disclosure comprise additional steps after binding protein subject to an above referenced wash step and has been eluted from an affinity chromatography column. For example, the eluted binding protein may be subsequently subject to additional chromatography steps. For example, the eluted binding protein may be subject to cation exchange chromatography to remove any potential high molecular weight species. For example, cation exchange chromatography may be used to remove high molecular weight aggregates such as binding protein complexes. Accordingly, in an example, the methods of the present disclosure may further comprises cation exchange chromatography. Other exemplary additional chromatography purification steps include but are not limited to ionic exchange chromatography, and/or hydrophobic interaction chromatography, and/or mixed mode chromatography and/or size exclusion chromatography. In one example, the methods of the present disclosure further comprise a viral inactivation step. In another example, the methods of the present disclosure further comprise a viral filtration step.

In an example, clarified cell culture fluid is purified to remove free light chain not associated with heavy chain according to the present disclosure before being subject to cation exchange chromatography, ultrafiltration, anion exchange chromatography, phenyl sepharose HP chromatography, viral filtration and ultrafiltration.

### Formulation

In an example, the method further comprises a step of formulating the purified binding protein into a pharmaceutical composition.

The disclosure herein further provides, for example, a pharmaceutical composition comprising a binding protein purified by a method as described herein.

An appropriate pharmaceutical composition comprising binding protein to be administered can be prepared in a physiologically acceptable carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. The compound can be lyophilized for storage and reconstituted in a suitable carrier prior to use according to art-known lyophilization and reconstitution techniques.

The optimum concentration of the active ingredient(s) in the chosen medium can be determined empirically, according to procedures known to the skilled artisan, and will depend on the ultimate pharmaceutical formulation desired.

Similarly, in an example, the binding proteins can be purified according to the present disclosure and formulated into a diagnostic composition comprising one or more of the above referenced components in accordance with the intended application of the composition (e.g. in vitro vs in vivo).

### EXAMPLES

### Example 1: Expression of Anti-kappa Myeloma Antigen (KMA) Binding Protein

Anti-kappa myeloma antigen (anti-KMA) binding protein is expressed by growing Chinese Hamster Ovary (CHO) cells genetically modified to express the binding protein in fed-batch suspension culture. Production of the genetically modified CHO cells used herein is generally described in WO2003/004056. Following suspension culture, CHO cells are removed from the cell culture fluid (CCF) by depth filtration and 0.2 μm filtration. The resultant CCF is collected and stored at 2° C.to 8° C.

### Example 2: Protein A Affinity Chromatography Purification Of Recombinant Anti-kappa Myeloma Antigen (KMA) Binding Protein

To prepare the CCF containing anti-KMA binding protein for protein A chromatography purification, the protein concentration of the CCF was adjusted to ≤3.5 g/L and pH of ≥6.8.

The protein A affinity chromatography column (MabSelect Xtra Load) was equilibrated with at least 5 column volumes (CVs) of 1× PBS (equilibration buffer) to a pH of 7.3 to 7.5 and conductivity between 14.0-16.8 mS/cm.

The protein concentration and pH adjusted CCF was then loaded onto the equilibrated Protein A affinity chromatography column before the column was washed with ≥15 CVs basic wash buffer of 200 mM sodium carbonate, 1M sodium chloride, pH 10.2. The column was further washed with ≥5 CVs of basic wash buffer of 100 mM sodium carbonate, pH 10.2 followed by ≥4 CVs of acidic wash buffer of 35 mM sodium phosphate, pH 6.2.

The anti-KMA binding protein was eluted from the protein A affinity chromatography column with an elution buffer of 10 mM sodium phosphate pH 3.0 and monitored by measuring the absorbance wavelength of 280 nm. A single peak fraction was collected which starts at 15% above and ends at 5% above the baseline, respectively.

### Example 3: Alternative Protein A Affinity Chromatography Purification of Recombinant Anti-KMA Binding Protein

In an alternative approach, the CCF was purified by protein A affinity chromatography with two washes of the basic wash buffer (200 mM sodium carbonate, 1M sodium chloride, pH 10.2) followed by elution and collection of anti-kappa myeloma binding protein from the protein A affinity chromatography column. Results were comparable to Example 2.

### Example 4: Low pH Treatment and Adjustment

The protein A affinity chromatography eluent containing anti-KMA binding protein was further subject to low pH viral inactivation, neutralisation and viral filtration. Viral inactivation was performed by adjusting the pH of the protein A affinity chromatography eluent to 3.40 to 3.60 with 1 N hydrochloric acid and holding the eluent at room temperature for 60 to 75 minutes to inactivate any potentially contaminating virus. Viral inactivation was followed by neutralisation of the eluent with 1N sodium hydroxide to a pH of 6.1 to 6.3 followed by 0.2 μm filtration and stored at 2° C. to 8° C. until further processing.

The yield of anti-KMA monomer following purification via example 2 or 3 and subsequent viral inactivation, neutralisation and filtration was 90% relative to anti-KMA binding protein bound to free light chain not associated with heavy chain as determined by SEC-HPLC and activity determined by BioCore assay.

### Example 4: Cation Exchange Chromatography Purification

Following low pH viral inactivation, neutralisation and viral filtration of the protein A affinity chromatography eluent, the eluent was subject to cation exchange chromatography to remove any potential high molecular weight species present in the eluent. The cation exchange chromatography column (Fractogel EMD SE HiCap) was equilibrated with ≥5 CVs of 35 mM sodium phosphate at pH 6.2. The protein concentration of the eluent was adjusted to ≤8.5 mg/ml pH 6.1 to 6.3 before loading onto the cation exchange chromatography column.

The anti-KMA binding protein bound to the cation exchange chromatography column was washed with ≥5 CVs of 20 mM sodium phosphate pH 6.2, and anti-KMA binding protein was eluted with 35 mM sodium phosphate and 30 mM sodium chloride pH 6.2 monitored by measuring the absorbance wavelength of 280 nm. A single peak fraction was collected above baseline and ending when absorbance descended to approximately 20% to 30% of the maximum peak height.

The combination of protein A affinity chromatography and cation exchange chromatography resulted in the recovery of 95% pure anti-KMA binding protein monomer relative to anti-KMA binding protein bound to free light chain not associated with heavy chain as determined by SEC-HPLC and activity determined by BioCore assay.

### Example 5: Formulation Into a Pharmaceutical Composition

The cation exchange chromatography eluent was passed through a Planova 20 N virus removal filter to remove any inadvertent viral contamination and further filtered through a 0.22 μm filter. The filtered preparation was concentrated and diafiltered by tangential flow filtration (TFF) into 20 mM sodium citrate, 100 mM sodium chloride, 1.5% mannitol, 50 μM DTPA pH 6.0 and further filtered through 0.2 μm filter. Polysorbate 80 (Tween 80) was added to a final concentration of 0.04% where required and protein concentration adjusted to 10.0±1.0 mg/ml and filtered through 0.2 μm filter to produce a formulated pharmaceutical composition of anti-KMA binding protein.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

The present application claims priority from Australian Provisional Patent Application 2020900948 filed 27 Mar. 2020, the entire contents of which are incorporated herein by reference.

1-41. (canceled)

42. A method for purifying an anti-kappa myeloma antigen (KMA) antibody that binds free kappa light chain not associated with heavy chain from a Chinese Hamster Ovary (CHO) cell culture comprising the anti-KMA antibody, the method comprising:
binding the antibody from the CHO cell culture to a Protein A resin of neutral pH;
washing the Protein A resin with a basic wash buffer having a pH of at least 2.5 to 5 above neutral pH to wash free kappa light chain not associated with heavy chain from the composition; and
eluting antibody bound to the Protein A resin with an elution buffer.

43. The method of claim 42, wherein the anti-KMA antibody preferentially binds KMA over free kappa light chain not associated with heavy chain.

44. A composition comprising anti-kappa myeloma antigen (KMA) binding proteins, wherein less than 20%, less than 15%, less than 10%, or less than 6% of the anti-KMA binding proteins in the composition are anti-KMA binding proteins in complex with free kappa light chain not associated with heavy chain.

45. The composition of claim 44, wherein the anti-KMA binding proteins comprise a VH region set forth in SEQ ID NO:1 and a VL region set forth in SEQ ID NO:3 or bind the same epitope of KMA as an antibody comprising a VH region set forth in SEQ ID NO:1 and a VL region set forth in SEQ ID NO:3.

46. The composition according to claim 44, wherein the binding proteins are produced by Chinese Hamster Ovary (CHO) cells, preferably wherein the anti-KMA binding proteins are antibodies.

47. The composition according to claim 44, wherein the anti-KMA binding proteins are purified from free kappa light chain not associated with heavy chain by a method comprising:
loading a composition comprising the anti-KMA binding proteins and free kappa light chain not associated with heavy chain onto an equilibrated affinity chromatography column of neutral pH to bind the anti-KMA binding proteins in the composition to the affinity chromatography column;
washing the affinity chromatography column with a basic wash buffer having a pH of at least 2.5 to 5 above neutral pH to wash free kappa light chain not associated with heavy chain from the composition; and
eluting the anti-KMA binding proteins bound to the affinity chromatography column with an elution buffer.

48. The composition of claim 47, wherein the composition comprising the anti-KMA binding proteins and free kappa light chain not associated with heavy chain is cell culture fluid obtained from the cell culture of CHO cells which express the anti-KMA binding proteins.

49. The method of claim 42, wherein the basic wash buffer has one or more of the following:
a pH of 9 to 11 or a pH of 9.5 to 10.5; or
0.1M to 0.2M sodium carbonate; or 1M sodium chloride.

50. The method of claim 42, wherein washing the affinity chromatography to wash free kappa light chain not associated with heavy chain comprises washing the affinity chromatograph column twice with a basic wash buffer.

51. The method of claim 50, wherein in the first wash the basic wash buffer comprises 0.2M sodium chloride and in the second wash the basic wash buffer comprises 0.1M sodium chloride.

52. The method of claim 51, wherein the basic wash buffers have the same pH.

53. The method of claim 42, wherein washing the affinity chromatography column comprises washing with an acidic wash buffer.

54. The method of claim 53, wherein the acidic wash buffer has a pH of 5.5 to 6.5.

55. The method of claim 53, wherein the acidic wash buffer comprises about 35 mM sodium phosphate.

56. The method of claim 42, wherein the elution buffer is acidic, or the elution buffer has one or more of the following:
a pH lower than the acidic wash buffer,
a pH of 2.5 to 3.5, or
10 mM sodium phosphate.

57. The method of claim 47, wherein the affinity chromatography column is a protein A chromatography column.

58. The method of claim 42, wherein the method further comprises one or more of the following:
a viral inactivation step;
a viral filtration step; or
a step of formulating the eluted binding protein into a pharmaceutical composition or diagnostic composition.

59. The method of claim 42, wherein the free kappa light chain not associated with heavy chain is a kappa light chain dimer.

60. The method of claim 42, wherein the molecular weight of the free light chain not associated with heavy chain is between 22 and 100 kD.

61. The method of claim 42, wherein the eluted binding proteins are subject to cation exchange chromatography to remove any potential high molecular weight species, and:
at least 75% unbound antibody relative to antibody bound to free light chain not associated with heavy chain as determined by SEC-HPLC and/or BioCore assay; or
at least 85% unbound binding protein relative to binding protein bound to free light chain not associated with heavy chain as determined by SEC-HPLC and/or BioCore assay; or
at least 90% unbound binding protein relative to binding protein bound to free light chain not associated with heavy chain as determined by SEC-HPLC and/or BioCore assay; or
between 85% and 95% unbound binding protein relative to binding protein bound to free light chain not associated with heavy chain as determined by SEC-HPLC and/or BioCore assay.


SEQUENCE LISTING (10 sequences)

SEQ ID NO: 1 - protein, 449 aa, Artificial Sequence
  KappaMab VH
EVQLQQSGAELVKPGASVKLSCTASGFNIKDTYMHWVKQRPEQGLEWIGRIDPANGNTKYDPKFQGKATIIADTSSNTAYLQLSSLTSEDTAVYYCARGVYHDYDGDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 5 aa, Artificial Sequence
  KappaMab epitope amino acid sequence
LDIKR

SEQ ID NO: 3 - protein, 214 aa, Artificial Sequence
  KappaMab VL
DIVMTQSQKFMSTSVGDRVSVTCKASQNVGTNVAWYQQKPGQSPKALIYSTSYRYSGVPDRFTGSGSGTDFTLTISNVQSEDLAEYFCQQYNSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 11 aa, Artificial Sequence
  VH CDR1
GFNIKDTYMHW

SEQ ID NO: 5 - protein, 16 aa, Artificial Sequence
  VH CDR2
RIDPANGNTKYDPKFQ

SEQ ID NO: 6 - protein, 10 aa, Artificial Sequence
  VH CDR3
GVYHDYDGDY

SEQ ID NO: 7 - protein, 11 aa, Artificial Sequence
  VL CDR1
KASQNVGTNVA

SEQ ID NO: 8 - protein, 7 aa, Artificial Sequence
  VL CDR2
STSYRYS

SEQ ID NO: 9 - protein, 9 aa, Artificial Sequence
  VL CDR3
QQYNSYPYT

SEQ ID NO: 10 - protein, 5 aa, Artificial Sequence
  KappaMab epitope 2 improved binding amino acid sequence
LEIKR